(19) United States
(12) Patent Application Publication
(10) Pub. No.: US 2011/0151548 A1
(43) Pub. Date: Jun. 23, 2011

(54) GREASE ABSORPTION SYSTEM AND METHOD

(75) Inventors: Robert V. Clapp, III, Fort Dodge, IA (US); Clayton Vaughan, Fort Dodge, IA (US)

(73) Assignee: Joe Harvey, Gurnee, IL (US)

(21) Appl. No.: 13/039,618

(22) Filed: Mar. 3, 2011

Related U.S. Application Data
(63) Continuation of application No. 11/935,101, filed on Nov. 5, 2007.

(51) Int. Cl. C12S 9/00; C12M 1/00

(52) U.S. Cl. 435/264; 435/283.1

(57) ABSTRACT

An improvement is provided in grease trap assemblies for grease traps to be positioned on the roof of commercial buildings such as restaurants. The improvement comprises a system for degreasing that uses, in combination, a grease absorbent material that is porous rock, preferably lava, and a grease digestible microbe capable of digesting fatty acids from cooking fats and oils. Each can as well be used alone, but best results are achieved with the combination.

### CROSS-REFERENCE TO RELATED APPLICATIONS

This application is a Continuation Application of U.S. Ser. No. 11/935,101 filed Nov. 5, 2007, herein incorporated by reference in its entirety.

### FIELD OF THE INVENTION

This invention relates generally to improvements in grease containment and/or absorption systems for collecting grease from discharged vents, usually on the roofs of commercial buildings, such as restaurants.

### BACKGROUND OF THE INVENTION

Restaurants and fast-food facilities do much cooking in grease. Typically the deep fat fryers have a hood which exhausts the vaporized grease away from the fryer via an exhaust fan and out to the roof area. However, the grease does deposit on the exhaust fan and also on the roof of the restaurant. Over a period of time this grease gradually builds up and collects about the fan structure, the fan housing, and as well on the roof. If not removed in time, the grease will become highly corrosive to the roofing materials, eventually causing deterioration of shingles and rotting of the underlying boards. Accumulated grease is also known to provide a significant fire hazard, and since it makes the surrounding area highly slippery, also a safety hazard.

When fatty acids from cooking fats, oils and grease come in contact with a roofing system it causes most roofs to soften, delaminate, blister or crack depending on the specific roof material. Fatty acids from grease void most roof warranties and roof replacements are expensive. Pooling grease and its runoff into the storm sewer presents an environmental problem and potentially costly fines. Also, fire inspectors can and will close a commercial business if they find excessive grease on the roof. There is, therefore, a continual need to prevent such grease accumulation so that the attendant problems associated therewith can be eliminated.

There have been previous roof top grease trap systems patented by others. One such attempt is disclosed in U.S. Pat. No. 5,196,040 entitled “Grease Trap and Filter Apparatus,” ('040 patent). However, the '040 patent has numerous shortcomings. For instance, the filter assembly is designed and cut to fit snugly around the exhaust duct of an exhaust system. However, the grease may still easily run in between the duct and the filter, spilling on to the roof.

U.S. Pat. No. 5,512,073 entitled “Grease Filter Assembly” ('073 patent) is another attempt to contain the grease that may be discharged through a roof top exhaust system. Similar to the above, the '073 patent also places the filter directly against the exhaust duct. However, the '073 patent is secured to the duct rather than the roof, allowing it to be placed on any roof, even if slanted.

U.S. Pat. No. 5,567,216 entitled “Grease Filter Assembly” ('216 patent) places a filter directed below the grease spout. However, it is well known that the grease has a tendency to expel from any openings in the seams around the entire perimeter of the exhaust duct, vent and under the fan base as well.

U.S. Pat. No. 6,010,558 entitled “Grease Containment System and Method for Absorbing Grease” ('558 patent) is directed to a system that uses a box to collect grease falling from the spout as well as a filter mat placed around the vent to collect run off grease.

U.S. Pat. No. 6,143,047 entitled “Effluent Containment Assembly” is directed to a containment system for collecting grease or other effluents that dispense from a exhaust system. The various disclosed systems may be mounted to the duct above the roof and include a flashing member to direct the effluent downwardly and outwardly away from the vent. However, since the filter material is cut and sized about the duct, grease may still seep in between the filter and the duct and onto the roof.

U.S. Pat. No. 6,468,323 includes an absorbent pad with a system that permits water to drain through or repel therefrom having a plurality of drain holes interspaced about the bottom portion such that water not absorbed or repelled by the absorbent pad may drain from the containment assembly.

All of the above systems are generally based upon the concept of containment. None are based on the concept of grease absorption and grease digestion. This latter system, i.e., grease absorption and digestion, is the combination of the present invention and as a result it provides longer life for absorption pads, substantially eliminates by bioremediation much of the grease and in general provides a much longer life system.

The combination of the present invention employs in the stacking arrangement above the grease barrier assimilation pad, a mixture of highly porous rock material, preferably lava, usually in combination with grease digestible microbes for bioremediation. The result is that the porous rock material, usually lava, absorbs a high percentage of the grease and its mixture with the microbes, which results in the microbes digesting a large portion of that grease. As a result, the grease barrier assimilation pad is exposed to much less grease over time than with the normal system. This results in containment, absorption, and grease digestion; results that are not collectively achievable by the prior art systems.

It is a primary objective of the present invention to achieve the above described three advantages, containment, absorption and digestion in the same system to allow more efficient grease removal with less hazard to the roof and the environment. The method of achieving this primary objective as well as others will become apparent from the following description of the invention.

### BRIEF SUMMARY OF THE INVENTION

An improvement in grease trap assemblies of grease traps to be positioned on the roof of commercial buildings such as restaurants is provided. The improvement comprises a system for degreasing that uses, in combination, a grease absorbent material that is porous rock, preferably lava, and a grease digestible microbe capable of digesting fatty acids from cooking fats and oils.

Numerous other advantages and features of the invention will become readily apparent from the following detailed description of the invention and the embodiments thereof, from the claims, and from the accompanying drawings.

### BRIEF DESCRIPTION OF THE DRAWINGS

FIG. 1 is a schematic illustration of the grease containment assembly in accordance with the present invention showing a typical grease exhaust system attached above the grease trap containment/absorption and digestion system of the present invention.

FIG. 2 is an exploded view showing the system in its entirety, in layers as would be typically installed.

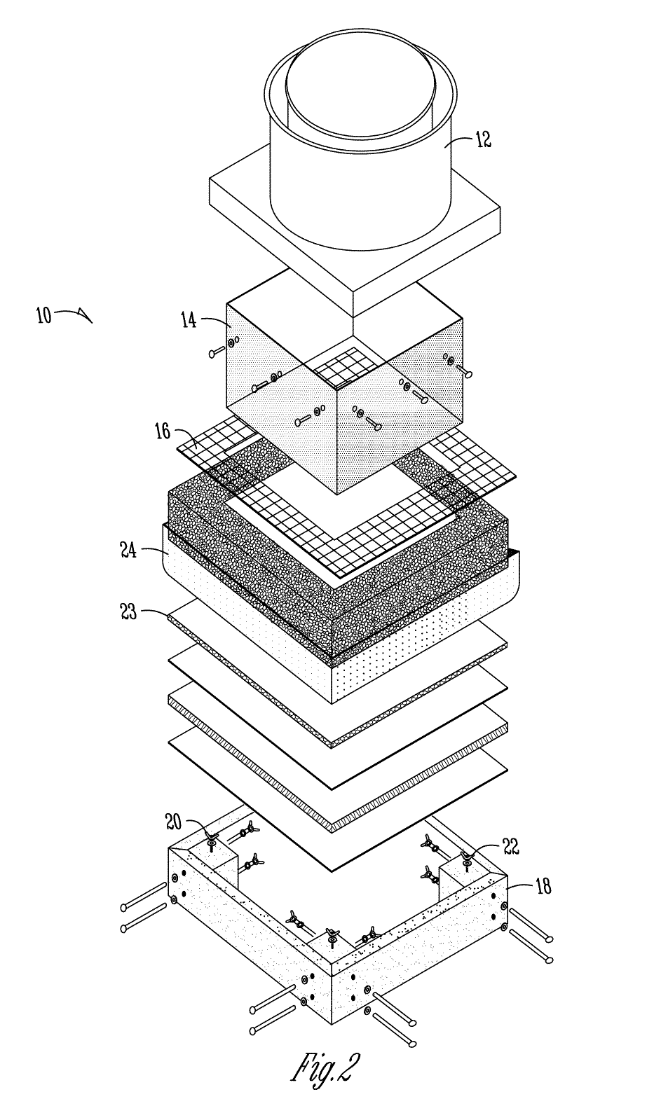

### DETAILED DESCRIPTION OF THE PREFERRED EMBODIMENT

While the invention is susceptible to embodiments in many different forms and subcombinations, there are shown in the drawings and will be described herein, in detail, the preferred embodiments of the present invention using both porous rock absorption and microbial bioremediation. It should be understood, however, that the present disclosure is to be considered an exemplification of the principles of the invention and is not intended to limit the spirit or scope of the invention and/or claims of the embodiments illustrated since one may use either porous rock alone or bioremediation alone and achieve some benefits.

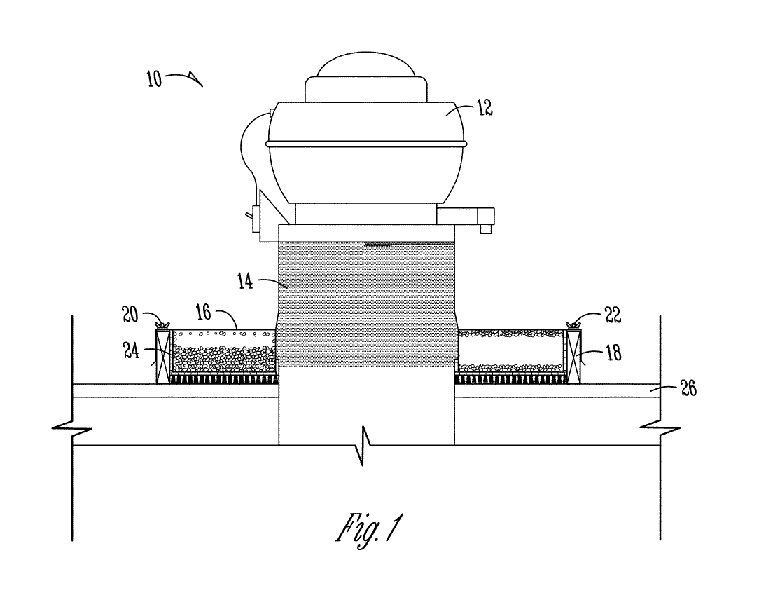

Looking first at FIG. 1, there is shown a grease containment absorption and digestion system 10 and its general component parts. Those parts will generally comprise an exhaust fan 12, grease deflector 14, both positioned on top of a containment screen 16 mounted to the grease box or frame 18 by screen anchor bolts 20 and 22. Positioned inside and around sides and across the bottom of frame 18 is grease barrier sheet 23 and assimilation pad 24. Underneath the frame 18 is drainage elevation board 26. These are the conventional elements of systems such as previously sold by our predecessor Rial, under U.S. Pat. No. 4,887,588. It also describes the typical systems mentioned in the background of the invention portion of the specification.

Typically the frame 18 is solid fire resistant and ultraviolet (UV) protected frame and is comprised of materials such as PVC that is weatherproof and will not rot, rust or mildew in any climate. Bolts 20 and 22 are preferably zinc treated to prevent corrosion.

The containment screen 16 is a strong nylon mesh capable of holding the porous rock material in place through the most turbulent conditions. Grease deflector 14 is a durable one-piece rubber ring that provides gap-free protection against runoff and directs all of the grease into the hereinafter described absorbent material. Coating the interior perimeter and bottom of the interior defined space of frame 18 is a grease barrier sheet 23 and assimilation pad 24. This is a fail-safe stopping point for any grease that finds its way through the porous material maze. These very dense woven sheets absorb and hold excess grease even when not maintained. They do, however, have the limits of capacity and the present invention improves the limits of their capacity, minimizing grease spill over onto the roof and thereby increasing the life of the entire system.

Placed on top of the absorbent pad and filling the rest of the space defined box or frame space 18 is the combination of absorption/digestion material of the present invention. The bulk of this material is preferably porous rock material. Especially suited for this use is porous lava rock. Subdivision is preferable because it increases the surface area for enhancing absorption. The preferable size of subdivided particles is between ½ inch and ¾ inch, preferably between ⅜ of an inch and ¾ of an inch in diameter.

While numerous rock or mineral materials might be suitable, the one found most suitable is porous lava rock. One highly suitable porous lava rock material is sold under the brand name Scoria. This porous lava rock soaks up grease like a sponge while allowing rain water to flow through unobstructed. It is also non-flammable so fire hazards are eliminated.

Preferably used in combination with the lava rock material is a suitable microbial bioremediation material. Such suitable bioremediation material includes those microbes capable of digesting fatty acids from cooking fats and oils and includes certain bacteria fungi and yeast. Commonly bacteria are employed.

Bacteria which are capable of hydrocarbon digestion are known. Biological agents are organisms that increase the rate at which natural biodegradation occurs. Biodegradation is a process by which microorganisms such as bacteria, fungi, and yeast break down complex compounds into simpler products to obtain energy and nutrients. Biodegradation of oil is a natural process that slowly—sometimes over the course of several years—removes oil from the aquatic environment.

Microbial Bioremediation technologies can help biodegradation processes work faster. Bioremediation refers to the act of adding materials to the environment, such as fertilizers or microorganisms, that will increase the rate at which natural biodegradation occurs. Two bioremediation technologies that are currently being used in the United States for oil spill cleanups are fertilization and seeding. These same technologies can be used for grease traps.

For further information concerning suitable bacteria for bioremediation see the patents of Anada Chakrabarty U.S. Pat. No. 5,535,061 entitled “Bacteria capable of Dissimilation of Environmentally Persistent Chemical Compounds”; and Chakrabarty, U.S. Pat. No. 4,259,444 entitled “Microorganisms having Multiple Compatible Degradative Energy-Generating Plasmids and Preparation Thereof”. The disclosure of each of these in terms of the bacteria capable of dissimilation of environmentally persistent compounds by one form or another of degradation is incorporated herein by reference. While the Chakrabarty patents describe pseudomonas microorganisms, there are many other naturally occurring harmless microorganisms which may also be used to degrade hydrocarbons such as fatty acids, fats and oils. Preferred ones are of course those which are harmless non-toxic and non-hazardous. The preferred strains are bacillus macerans (ATCC 202132), bacillus amyloliquefaciens (ATCC 202133), bacillus amyloliquefaciens (ATCC 202134), bacillus macerans (ATCC 202135), bacillus pumilus (ATCC 202136), and bacillus subtillis (ATCC 202137, ATCC 202138, ATCC 202139).

Suitable bioremediation cleaners for use in the grease trap systems of the present invention are known and commercially available. For example such cleaners can be obtained from Dawg, Inc., 25 Lassy Court, Terryville, Conn. 06786. The amount used can be within the manufacturers expressed literature for their products. But generally if one treats a kilogram of rock with 1.0×10⁸ cfu's then enough has been applied to consume the grease. In regard to a range of application, from about 500,000 cfu/1 cg to about 300 billion cfu/kg of rock provide some efficacious effect. The microbial can be added in a concentrated dry or liquid form. But experience has proven better adherence to the rock bed of the grease trap is achieved with a liquid delivery system. The rock may be treated with microbes before making the grease trap or after assembling the system on the roof. It also may be treated via spray to replenish the microbes from time to time.

From the foregoing and as mentioned above, it will be observed that numerous variations and modifications may be effected without departing from the spirit and scope of the novel concept of the invention. It is to be understood that no limitation with respect to the specific methods and apparatus illustrated herein is intended or should be inferred. It is, of course, intended to cover by the appended claims all such modifications as fall within the scope of the claims.

What is claimed is:

1. A rooftop grease absorption/digestion system, comprising;
a grease box frame,
a grease barrier sheet, within the frame and for lying on the rooftop,
an absorption pad within the frame, on top of said grease barrier sheet;
an exhaust fan mounted to said roof within the grease box frame;
said exhaust fan having a grease deflector positioned under it and above said grease box frame;
said grease box being filled with subdivided grease absorption and/or digestion material.

2. The grease box of claim 1 which includes a fire resistant grease box.

3. The grease box of claim 2 which includes an ultraviolet light resistant grease box.

4. The grease box of claim 3 which is made from polyvinyl chloride.

5. The grease box of claim 1 which includes a drainage elevation board under said grease deflector.

6. The grease box of claim 1 which includes a mesh screen over said subdivided grease absorption and/or digestion material.

7. The grease absorption assembly of claim 1 wherein the grease absorption material is porous rock.

8. The grease absorption assembly of claim 2 wherein the grease absorption material is subdivided lava rock.

9. The grease box of claim 1 which has both grease absorption and digestion capability.

10. The grease box of claim 9 which includes both porous granular absorption material and grease digestion microbes selected from the group consisting of bacteria, fungus and yeasts.

11. The grease absorption assembly of claim 10 wherein the microbes are bacterial.

12. The grease absorption assembly of claim 10 wherein the microbes are selected from the bacteria commonly used for microbial bioremediation of hydrocarbons.

13. A method of controlling grease from collecting on the roofs at restaurant buildings and the like comprising:
building a grease absorption assembly for positioning around the perimeter of a grease exhaust system on the roof of said building;
placing within a grease absorption assembly porous subdivided lava rock to absorb grease; and
treating the lava rock with grease digestible microbes at a level sufficient to form from about 500,000 cfu/kg of lava rock to 300 billion cfu/kg of lava rock to provide grease digestion and microbial remediation of the acids from cooking fats and oils.
